(19) United States
(12) Patent Application Publication
(10) Pub. No.: US 2012/0059457 A1
(43) Pub. Date: Mar. 8, 2012

(54) MINIMALLY INVASIVE SURGICAL INSTRUMENT FOR DELIVERY OF CARDIAC DEVICES

(76) Inventors: Karl R. LEINSING, Hampton, NH (US); JaiShankar Raman, Chicago, IL (US)

(21) Appl. No.: 12/874,277

(22) Filed: Sep. 2, 2010

(51) Int. Cl. A61F 2/24

(52) U.S. Cl. 623/2.11

(57) ABSTRACT

A minimally invasive surgical instrument for delivering an external basal annuloplasty device to a heart includes a hollow shaft with a telescopically center tube extending from an end of the hollow shaft. The center tube includes a heart gripping member. A plurality of delivery prongs are slidably coupled to the hollow shaft, each having a distal portion with an expandable wire member for holding the external basal annuloplasty device. A sleeve is telescopically coupled to the expandable wire member. A wire slide is coupled to a proximal portion of the expandable wire member and a sleeve slide is coupled to a proximal portion of the sleeve. The external basal annuloplasty device is mountable on the plurality of delivery prongs by inserting each expandable wire member into a respective pocket on the external basal annuloplasty device.

### FIELD

The following description relates generally to surgical instruments and more particularly to minimally invasive surgical instruments for delivery of cardiac devices.

### BACKGROUND

Dilatation of the base of the heart occurs with various diseases of the heart and often is a causative mechanism of heart failure. In some instances, depending on the cause, the dilatation may be localized to one portion of the base of the heart (e.g., mitral insufficiency as a consequence of a heart attack affecting the inferior and basal wall of the left ventricle of the heart), thereby affecting the valve in that region. In other cases, such as cardiomyopathy, the condition may be global affecting more of the heart and its base, causing leakage of particularly the mitral and tricuspid valves. Other conditions exist where the mitral valve structure is abnormal, predisposing to leakage and progressive dilatation of the valve annulus (area of valve attachment to the heart). This reduces the amount of blood being pumped out by the ventricles of the heart, thereby impairing cardiac function further.

In patients with heart failure and severe mitral insufficiency, good results have been achieved by aggressively repairing mitral and/or tricuspid valves directly, which requires open-heart surgery (Bolling, et al). The mitral valve annulus is reinforced internally by a variety of prosthetic rings (Duran Ring, Medtronic Inc) or bands (Cosgrove-Edwards Annuloplasty Band, Edwards Lifesciences Inc). The present paradigm of mitral valve reconstruction is therefore repair from inside the heart, with the annulus being buttressed or reinforced by the implantation of a prosthetic band or ring. Since this is major open-heart surgery with intra-cavitary reconstruction, there is the attendant risk of complications and death associated with mitral valve surgery. Another approach has been to replace the mitral valve, which while addressing the problem, also requires open-heart surgery and involves implantation of a bulky artificial, prosthetic valve with all its consequences. Because every decision to perform major surgery requires some risk vs. benefit consideration, patients get referred for risky surgery only when they are significantly symptomatic or their mitral valve is leaking severely.

In contrast to the more invasive approaches discussed above, in specific instances of inferior left ventricular wall scarring causing mitral regurgitation, Liel-Cohen and co-workers have suggested localized pressure or support of the bulging scar of the inferior wall of the heart from the outside (Liel-Cohen. N. et al. (2000) “Design of a new surgical approach for ventricular remodeling to relieve ischemic mitral regurgitation: insights from 3-dimentsional echocardiography”. Circulation 101 (23):2756-2763).

Another less invasive approach to preventing global heart dilation is ventricular containment with a custom made polyester mesh, or cardiac support device (U.S. Pat. Nos. 6,077,218 and 6,123,662). These devices are designed to provide a passive constraint around both ventricles of the heart, and constrain diastolic expansion of the heart. Other devices include ventricular assist devices that provide cardiac assistance during systole and dynamic ventricular reduction devices that actively reduce the size of the heart. However, this technique does not specifically address valve leakage using a device that reinforces the base of the heart in all phases of the cardiac cycle.

Another less invasive approach is found in U.S. Pat. No. 6,716,158 to Raman et. al. However, although the Raman et. al. system operates to stabilize the base of the heart, there is also the need for a system to modulate or modify heart valve function by applying localized pressure to particular regions of the heart; for example, to tissues adjacent to heart valve. A system that satisfies this need is disclosed in U.S. Patent Application Publication No. 2009/0062596 to Leinsing et al., which is incorporated herein by reference as if set forth verbatim. This system, which includes a surgical procedure termed basal annuloplasty of the cardia externally (BACE™), is herein after referred to as the BACE system. The BACE system advantageously applies inward pressure to tissue adjacent to the heart valves so as to modify the shape or reduce the size of a heart valve itself. In particular, the BACE system can be used to repair or re-configure the shape of a mitral and/or tricuspid valve so as to treat valve dilation and resulting valve insufficiency problems.

Although the BACE system provides a less invasive, simple technique of repairing, reinforcing, reducing or stabilizing the base of the heart and its underlying valves (mitral and tricuspid valves) from the outside, there remains a need for an apparatus and method for delivering the BACE device to the heart in a minimally invasive manner. In particular, there is a need for an apparatus and method for delivering the BACE device to the heart through a relatively small incision in the abdomen so that open heart surgery can be avoided.

Apparatuses for delivering cardiac devices to the heart in a minimally invasive manner are known. For example, U.S. Pat. No. 7,651,462 to Hjelle et al. discloses an apparatus for placing a cardiac support device (CSD) on a heart, The Hjelle apparatus includes a plurality of finger-like “release elements” which actively engage with the CSD and which are disengaged from the CSD in order to release the CSD onto the heart. However, the active release elements of the Hjelle apparatus are relatively complex and not specifically designed for use with the BACE device. There is a need for an apparatus for delivering the BACE device to the heart in a minimally invasive manner, and that passively holds the BACE device using only friction rather than actively engaging with the BACE device with release element. Further, there is a need for such a cardiac delivery apparatus that gives the surgeon the ability to precisely position the BACE device on the heart, and to ensure that the BACE device remains in the proper location on the heart when the BACE device is being released from the cardiac delivery apparatus.

The presently disclosed embodiments are directed to apparatuses and methods for solving the above mentioned problems. The presently disclosed embodiments can advantageously be used to deliver the BACE device to the heart using minimally invasive surgical techniques.

### SUMMARY

The following simplified summary is provided in order to provide a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented later.

In one aspect of the disclosed embodiments a minimally invasive surgical instrument for delivering an external basal annuloplasty device to a heart is provided. The surgical instrument includes a hollow shaft having a proximal portion and a distal portion. A center tube is telescopically coupled to the hollow shaft and extends from the distal portion of the hollow shaft. The center tube has a heart gripping member at its distal end. A plurality of delivery prongs are slidably coupled to the hollow shaft. Each delivery prong includes an outwardly biased distal portion with an expandable wire member for holding the external basal annuloplasty device. A sleeve is telescopically coupled to the expandable wire member. A wire slide is coupled to a proximal portion of the expandable wire member and a sleeve slide is coupled to a proximal portion of the sleeve. The external basal annuloplasty device is mountable on the plurality of delivery prongs by inserting each expandable wire member into a respective pocket on the external basal annuloplasty device.

In some embodiments, the wire slide and sleeve slide of each delivery prong are releasably coupled to each other so that the external basal annuloplasty device is releasable from the plurality of delivery prongs by decoupling each sleeve slide from each respective wire slide and withdrawing each expandable wire member out of each respective pocket on the basal annuloplasty device and into each respective sleeve. The expandable wire member of each delivery prong may be an elongate flexible wire loop at a distal end of a pair of substantially parallel posts with proximal ends coupled to the wire slide. The elongate flexible wire loop increases in width when the elongate flexible wire loop is extended out of the sleeve and decreases in width when the elongate flexible wire loop is withdrawn into the sleeve.

In some embodiments, the heart gripping member is a suction cup. For example, the center tube may be hollow and connected to a vacuum source to provide a vacuum to the suction cup.

Prior to delivery of the external basal annuloplasty device to the heart, the hollow shaft constrains the outwardly biased distal portions of the delivery prongs into a compact configuration substantially parallel to the hollow shaft. Sliding each of the wire slides and sleeve slides distally along the hollow shaft causes the outwardly biased distal portions of the delivery prongs to outwardly expand into a deployment configuration. A containment sheath may be connected to the distal portion of the hollow shaft for containing the external basal annuloplasty device prior to delivery.

In some embodiments, the surgical instrument includes an external basal annuloplasty device mounted on the plurality of delivery prongs. The external basal annuloplasty device includes a plurality of pockets each receiving respective expandable wire members of the plurality of delivery prongs. The external basal annuloplasty device may include a band dimensioned to be received around a patient's heart, the band having an inner layer and an outer layer, wherein some but not all areas of the inner layer and outer layer are bound to one another. The band of the external basal annuloplasty device may also include at least two fillable chambers, the at least two fillable chambers being located in areas where the inner layer and the outer layer are not bound to one another. The at least two fillable chambers are positioned spaced apart from one another so as to form a bridge over vasculature on the heart when the at least two fillable chambers are filled.

A method of using the surgical instrument described above to deliver an external basal annuloplasty device to a heart in a minimally invasive surgical procedure is also provided. First the surgical instrument described above and an external basal annuloplasty device having a plurality of pockets are provided. The external basal annuloplasty device is mounted on the surgical instrument by inserting each of the plurality of expandable wire members of the surgical instrument into respective pockets of the external basal annuloplasty device. The heart is then gripped with the heart gripping member of the surgical instrument. The plurality of delivery prongs are slid distally along the hollow shaft causing the outwardly biased distal portions of the delivery prongs and the external basal annuloplasty device to expand into a deployment configuration. The external basal annuloplasty device is aligned with a predetermined location on the heart and then the external basal annuloplasty device is deployed onto the heart by removing the plurality of expandable wire members of the surgical instrument from the respective pockets of the external basal annuloplasty device. The plurality of expandable wire members of the surgical instrument are removed from the respective pockets of the external basal annuloplasty device by decoupling each wire slide from each respective sleeve slide and sliding each wire slide proximally to withdraw each expandable wire member inside each respective sleeve.

To the accomplishment of the foregoing and related ends, certain illustrative aspects are described herein in connection with the following description and the annexed drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings.

### BRIEF DESCRIPTION OF THE DRAWINGS

FIG. 1 is a perspective view of one embodiment of a minimally invasive surgical instrument.

FIG. 2 is a perspective view of the distal end of the minimally invasive surgical instrument of FIG. 1.

FIG. 3 is a cutaway perspective view of the proximal end of the minimally invasive surgical instrument of FIG. 1.

FIG. 4 is a cutaway perspective view of the proximal end of the minimally invasive surgical instrument of FIG. 1, with sleeve slide members not shown.

FIG. 5 is a cross-sectional view of the proximal end of the minimally invasive surgical instrument of FIG. 1.

FIG. 6 is a perspective view of one embodiment of an external basal annuloplasty device for use with the minimally invasive surgical instrument of FIG. 1.

### DETAILED DESCRIPTION

In one aspect of the disclosed embodiments, a minimally invasive surgical instrument is used to deliver an external basal annuloplasty device to the heart for mitral valve repair. In particular, the minimally invasive surgical instrument may be used for delivery of the BACE device to heart. The instrument includes a hollow shaft telescopically coupled to a center tube with the center tube inside the hollow shaft and extending out the distal end of the shaft. A plurality of delivery prongs are slidably coupled to the hollow shaft and each have an expandable wire member at their distal end. The expandable wire members passively frictionally hold the external basal annuloplasty device, and in particular the expandable wire members are inserted into pockets on the BACE device. The expandable wire members are telescopically coupled inside of sleeves so that the expandable wire members can be removed from frictional contact with the external basal annuloplasty device by sliding the expandable wire members proximally until they are completely withdrawn inside the sleeves.

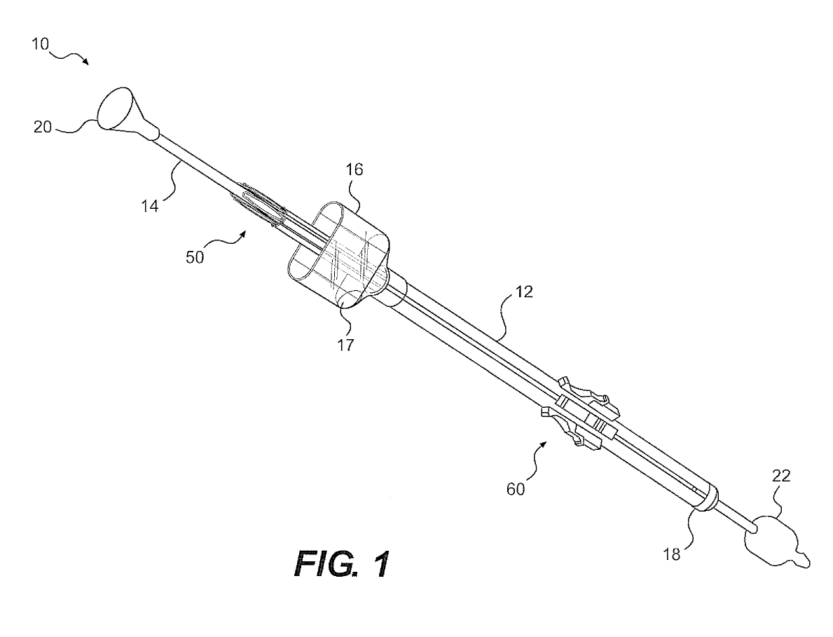

FIG. 1 shows a first embodiment of a minimally invasive surgical instrument 10 for delivery of an external basal annuloplasty device to a heart. Instrument 10 includes hollow shaft 12 which is telescopically coupled to center tube 14, which extends from the distal end of hollow shaft 12. As used herein, the term “telescopically coupled” means that two or more elongate members are slidably coupled to one another with one of the elongate members inside of the other. Furthermore, the terms proximal and distal are in reference to the perspective of the surgeon using instrument 10. Thus, the proximal end of instrument 10 is the end which is closest to the surgeon, and the distal end is the end which is furthest from the surgeon.

Containment sheath 16 is attached to the distal end of hollow shaft 12 and is used to contain the external basal annuloplasty device prior to deployment onto the heart. In the illustrated embodiment center tube 14 is hollow and also extends from the proximal end of hollow shaft 12 through end cap 18, although this is not necessarily the case in other embodiments. A heart gripping member such as suction cup 20 is connected to the distal end of center tube 14. Suction cup 20 is in fluid communication with a vacuum source (not shown) connected to ball knob 22 at the proximal end of center tube 14. A plurality of delivery prongs 50 are slidably coupled to hollow shaft 10. As will be explained in greater detail below, the external basal annuloplasty device is passively frictionally held by delivery prongs 50 prior to delivery of the device to the heart. Finally, slide pairs 60 are connected to the proximal ends of delivery prongs 50. The purpose of slide pairs 60 will be described in greater detail below.

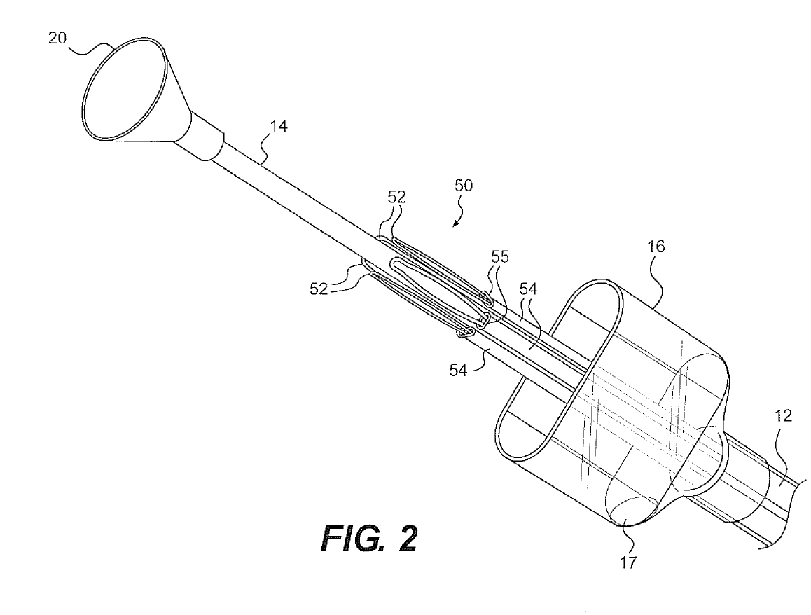

FIG. 2 shows the distal end of instrument 10 in more detail, with particular focus on delivery prongs 50. In the illustrated embodiment, five delivery prongs 50 are shown. However, it is to be understood that a smaller or larger number of delivery prongs 50 is within the scope of the present disclosure. Each delivery prong 50 includes expandable wire member 52 at its distal end. Each expandable wire member 52 is telescopically coupled to respective sleeves 54 so that expandable wire member 52 may be slid into or out of the distal end of sleeve 54. Each expandable wire member 52 is flexible and outwardly bowed. Thus, when expandable wire member 52 is withdrawn inside sleeve 54, its width is constrained by sleeve 54. When expandable wire member 52 is slid out the distal end of sleeve 54, expandable wire member 52 is no longer constrained and therefore increases in width due to its outwardly bowed bias. In the illustrated embodiment expandable wire member 52 is a flexible wire loop with a generally elliptical shape. However, other shapes of expandable wire member 52 are also within the scope of the present disclosure. The distal ends 55 of sleeves 54 are rounded to eliminate any sharp edges that could damage the external surface of the heart during surgery.

Expandable wire member 52 may be the distal end of a pair of substantially parallel guide wires 58 which extend proximally to connect to each slide pair 60. Thus, each delivery prong may be considered to include expandable wire member 52, parallel guide wires 58, sleeve 54, and slide pair 60. Furthermore, each delivery prong 50 may be outwardly biased. For example, either expandable wire member 52, parallel guide wires 58, or sleeve 54 may be naturally biased to spread radially outward relative to center tube 14 if not otherwise constrained, for example by hollow shaft 12. However, in other embodiments the delivery prongs 50 are not naturally outwardly biased. For example, delivery prongs 50 may be flexible but naturally straight. In order to cause delivery prongs 50 to spread outward, delivery prongs 50 may be slid distally along center tube 14 and then deflected by an outwardly tapered object such as suction cup 20.

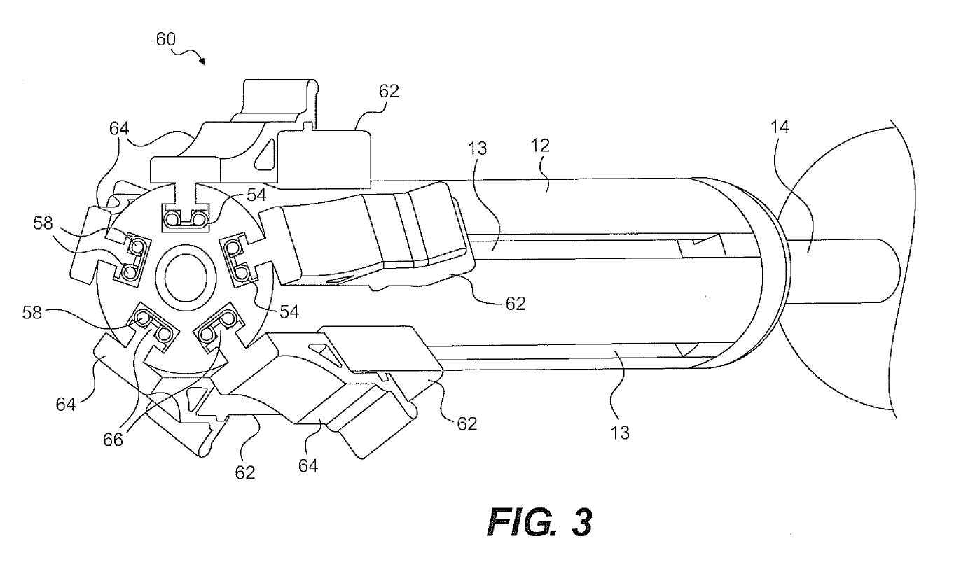

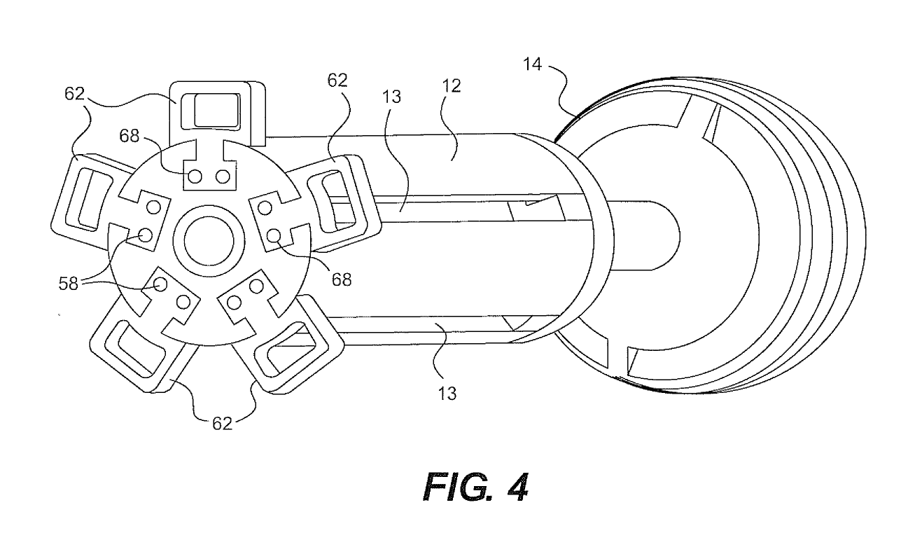

FIGS. 3 and 4 show cutaway views of instrument 10 with particular focus on slide pairs 60. Each slide pair 60 is coupled to the proximal end of a respective delivery prong 50 and may therefore be considered the most proximal component of each delivery prong 50. Each slide pair 60 is slidably engaged with a respective longitudinal slot 13 of hollow shaft 12. Each slide pair 60 includes wire slide 62 and sleeve slide 64. Each wire slide 62 is fixedly connected to the proximal ends of parallel guide wires 58. In other embodiments, parallel guide wires 58 may be replaced by another elongate member connected to expandable wire member 52, such as an elongate strip. In any case, wire slide 62 is fixedly connected to the proximal end of the elongate structure which is connected at its distal end to expandable wire member 52.

As best seen in FIG. 3, each set of parallel guide wires 58 and sleeve 54 extend to each slide pair 60. Each set of parallel guide wires 58 is telescopically mounted within a respective sleeve 54. It can thus be seen that there is a small space between parallel guide wires 58 inside sleeve 54. This small space accommodates one or more pins 66 in each sleeve slide 64 which extend through corresponding small apertures in sleeve 54 so that pins 66 extend into the small space between parallel guide wires 58 inside sleeve 54. These pins 66 therefore serve to couple sleeve slide 64 to sleeve 54. Sliding sleeve slide 64 along longitudinal slot 13 of hollow shaft 12 results in corresponding movement of sleeve 54 along hollow shaft 12.

FIG. 4 shows a cutaway view of instrument 10 taken at a cross-section immediately proximal to the proximal ends of sleeve slides 64 shown in FIG. 3. In FIG. 4 it can be seen that parallel guide wires 58 extend into holes 68 in wire slide 62. Guide wires 58 are fixedly attached to wire slide 62 and may be bonded to wire slide 62 inside holes 68. Thus, sliding wire slide 62 along longitudinal slot 13 of hollow shaft 12 results in corresponding movement of parallel guide wires 58 and expandable wire member 52 along hollow shaft 14.

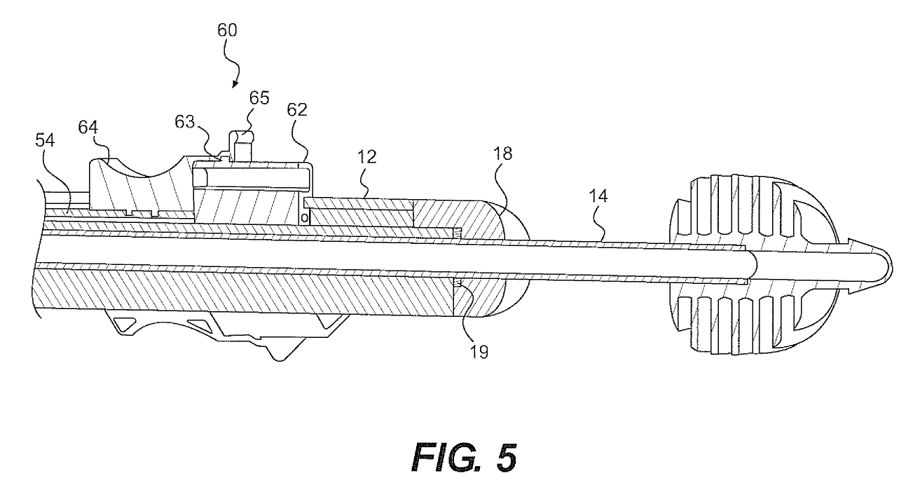

FIG. 5 shows a longitudinal cross-sectional view of the proximal end of instrument 10, in particular the coupling between wire slide 62 and sleeve slide 64. Wire slide 62 has a small protrusion on its outer radial surface, such as ridge 63. Sleeve slide 64 includes tab 65 at its proximal end. Ridge 63 of wire slide 62 is engageable with a complementary indent on the underside of tab 65 of sleeve slide 64. In FIG. 5, wire slide 62 and sleeve slide 64 are shown coupled together so that wire slide 62 moves in tandem with sleeve slide 64. In other words, if either wire slide 62 or sleeve slide 64 is slid along longitudinal slot 13 of hollow shaft 12, then expandable wire member 52, parallel guide wires 58 and sleeve 54 will all correspondingly slide along hollow shaft 12.

To decouple wire slide 62 from sleeve slide 64, radial and distal pressure is applied to tab 65 of sleeve slide 64. This causes tab 65 to flex outwardly so that ridge 63 of wire slide 62 is clear of the indent in the underside of tab 65. Once tab 65 is flexed in this manner, wire slide 62 can be decoupled from sleeve slide 64 by sliding wire slide 62 proximally and/or by sliding sleeve slide 64 distally a small amount. Once decoupled, wire slide 62 and sleeve slide 64 can slide along hollow shaft 12 independently of one another. Consequently, when wire slide 62 is decoupled from sleeve slide 64, expandable wire member 52 and parallel guide wires 58 can slide along hollow shaft 14 independently of sleeve 54. This ability to move expandable wire member 52 independently of sleeve 54 provides significant advantages to instrument 10 when the external basal annuloplasty device is being placed on the heart, as will be explained in greater detail below.

FIG. 5 also shows that in some embodiments the proximal end of center tube 14 extends from the proximal end of hollow shaft 12. O-ring 19 is secured in place at the proximal end of hollow shaft 12 by end cap 18 which is secured to hollow shaft 12 by an adhesive or by a threaded-fit, press-fit, or other suitable attachment mechanism. The inner radial surface of o-ring 19 contacts the outer radial surface of center tube 14. This contact between o-ring 19 and center tube 14 increases the frictional resistance to sliding movement of center tube 14 relative to hollow shaft 12. This added resistance is valuable because it reduces unwanted movements and ensures that hollow shaft 12 and center tube 14 can be moved smoothly and precisely with respect to each other, thereby facilitating accurate placement of the external basal annuloplasty device on the heart.

Also at the proximal end of instrument 10 is ball knob 22. Ball knob 22 includes internal bore 26 and is mounted onto the proximal end of center tube 14. Ball knob 22 may be connected to center tube 14 by adhesives, threaded engagement, press-fit engagement, or any other suitable method. Once mounted on center tube 14, the internal bore 26 of ball knob 22 is in fluid communication with the internal bore of center tube 14 and therefore also with suction cup 20. Ball knob 22 also includes barb fitting 24 for connecting with the hose of a vacuum source (not shown). Ball knob 22 is therefore used both as a handle for the surgeon to manipulate instrument 10 as well as a device for connecting instrument 10 to a vacuum source.

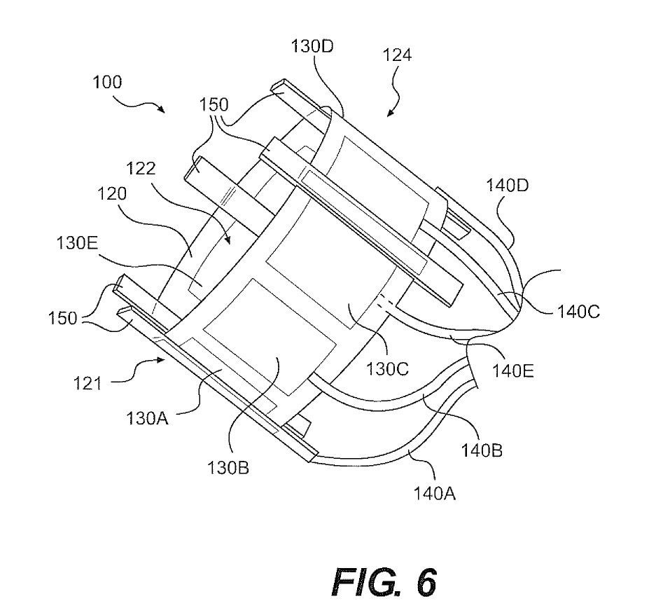

The process of using instrument 10 to place an external basal annuloplasty device on a heart will now be described. First, however, it will be instructive to describe an exemplary external basal annuloplasty device ideally suited for implantation by instrument 10. This exemplary external basal annuloplasty device, herein after referred to as BACE device 100, is shown in FIG. 6. BACE device 100 comprises a band 120 dimensioned to be received around a patient's heart. Band 120 comprises an inner layer 122 and an outer layer 124. Some areas of inner layer 122 and outer layer 124 are bound to one another, resulting in a very thin system design as can be seen. A unique advantage of such a thin band 120 is that it can easily be placed around the patient's heart during surgery.

Band 20 further comprises at least one fillable chamber 30 integrally formed therein. As depicted in FIG. 6, the band comprises five finable chambers 130A-E. Specifically, fillable chambers 130 are located in areas where inner layer 122 and the outer layer 124 are not bound to one another. Fillable chambers 130 may be integrally formed into band 120 when inner layer 122 and outer layer 124 are selectively bound together to create an enclosure. Each fillable chamber 130A-E is in fluid communication with a respective filling tube 140A-E. Two of the plurality of fillable chambers 130 may be positioned spaced apart from one another, such that band 120 has a gap 121 between chambers 130A and 130B which forms a bridge between the fillable chambers 130 when applied to a patient's heart. Preferably, band 120 and fillable chambers 130A and 130B are dimensioned such that the gap 121 is dimensioned to be positioned over vasculature on the exterior of the heart when fillable chambers 130A and 130B are filled thus forming a bridge therebetween. Thus, fillable chambers 130A and 130B can be positioned on opposite sides of the pulmonary trunk of the heart. Bridges can also be formed between 130B and 130C, 130C and 130D, and 130D and 130E. An important advantage of these bridges is that they do not need to form a space between the heart and the band. Instead, they only need to reduce localized pressure so as to prevent vascular occlusion. A bridge or release of pressure can also be formed by filling only one chamber. Filling only one chamber creates pressure directly under that chamber, but it also relieves pressure directly on each side of that chamber.

Filing tubes 140 may be made of silicone, or other suitable material. Each filing tube 140 is in fluid communication with, and fills, its own dedicated fillable chamber 130. For example, as depicted in FIG. 6, filling tube 140A fills finable chamber 130A, etc. It is to be understood that the present invention is not limited as to any particular substance being used for filing fillable chambers 130. As such, the individual tillable chambers 130 may be filled with substances including, but not limited to, a saline solution, a hardening polymer, a gel, or even a gas. Moreover, it is also to be understood that different tillable chambers 130 may be filled with different substances from one another. In various embodiments, the separate filling tubes 140 may be fillable through a blunt needle port 144 (for receiving blunt needle), a sharp needle port, or through a subcutaneous port. As such, different filling tubes 140 may be fitted with different ports at their point of connection with fillable chambers 130.

In one exemplary embodiment, band 120 is formed from silicone rubber and is therefore transparent. However, BACE device 100 is not so limited. For example, it is to be understood that band 120 may also be formed from other suitable biocompatible implantable materials, including, but not limited to a textile made from polyester, PTFE (polytetrafluoroethylene), or elastic yarns. An advantage of forming band 120 (and its tillable chambers 130) from a transparent material is that it facilitates accurate placement of the device around the patient's heart. In particular, the external vasculature of the heart is clearly viewable through band 120 as band 120 is placed around the patient's heart. Moreover, the transparent nature of the material permits easy positioning of fillable chambers 130 at preferred locations adjacent to heart valves (e.g., the mitral and/or tricuspid valve), and away from the vasculature.

In the embodiment of BACE device 100 shown in FIG. 6, BACE device 100 also includes a plurality of pockets 150 disposed on the outer surface of outer layer 124 of band 120. Pockets 150 may optionally be made of polyester, or any other suitable material, including, but not limited to other woven, knitted, non-woven, or other textiles. Pockets 150 in some embodiments act as promoters of controlled tissue growth such that they become secured to selected areas of the heart, but they may also act to limit tissue growth. Furthermore, pockets 150 may simply provide mechanical means of attachment. Pockets 150 may optionally be produced by molding them directly into band 120, or may be fitted onto band 120 by sutures or staples. It is to be understood that the term “pocket” does not exclude a sleeve or tube of material that is open at both ends. Rather, a “pocket” is any tubular (though not necessarily cylindrical) segment of material that can snugly receive an object inserted inside it, regardless of whether or not the pocket is open-ended at both ends.

The process of delivering BACE device 100 to a patient's heart using instrument 10 will now be described with reference to FIGS. 1-6. It is to be understood that the steps recited below are not necessarily performed in the order recited. Rather, the order of some steps may be rearranged, and some steps may be performed simultaneously with others. First, BACE device 100 is placed onto delivery prongs 50 of instrument 10. This is accomplished by ensuring that each expandable wire member 52 is extended out the distal end of its respective sleeve 54. If any expandable wire member 52 is withdrawn inside its respective sleeve 54, then the respective wire slide 62 is slid distally along hollow shaft 12 (or the respective sleeve slide 64 is slid proximally along hollow shaft 12) until expandable wire member 52 completely emerges from the distal end of sleeve 54. Once all of the expandable wire members 52 are appropriately extended from the distal ends of sleeves 54, any or every wire slide 62 may be coupled to its respective sleeve slide 64 by pressing them together until tab 65 of sleeve slide 62 pops over and engages ridge 63 of wire slide 62. In any delivery prong 50 in which wire slide 62 and sleeve slide 64 are coupled together, the respective expandable wire member 52, parallel guide wires 58, and sleeve 54 will all slide together along hollow shaft 12 in unison.

To mount BACE device 100 on delivery prongs 50, each expandable wire member 52 is inserted into one of the plurality of pockets 150 on the outer surface of BACE device 100. In an exemplary embodiment, the width of each pocket 150 is slightly smaller than the maximum width of expandable wire member 52. Thus, to insert expandable wire member 52 into pocket 150, expandable wire member 52 may have to be manually compressed so that it will fit inside pocket 150. Once expandable wire member 52 is inserted the desired depth into pocket 150, any manual compressive force on expandable wire member 52 is released so that expandable wire member 52 expands to snugly fit inside and make frictional contact with the inner surface of pocket 150. Ideally, the distal end of sleeve 54 should be adjacent, but not inside, the entrance of pocket 150. This process is repeated until each expandable wire member 52 is inserted inside a corresponding pocket 150 of BACE device 100.

Once BACE device 100 is mounted on delivery prongs 50 of instrument 10, BACE device 100 is withdrawn inside containment sheath 16. This is accomplished by threading filling tubes 140 of BACE device 100 through conduit aperture 17 of containment sheath 16 to provide more room inside containment sheath 16. Filling tubes 140 are thereby made easily accessible for connection to a fluid source to fill Tillable chambers 130. To completely withdraw BACE device 100 inside containment sheath 16, hollow shaft 12 is slid distally relative to delivery prongs 50 (or delivery prongs 50 are slid proximally relative to hollow shaft 12) until BACE device 100 is adjacent the proximal end of containment sheath 16. Containment sheath 16 helps to hold BACE device 100 in a compact configuration prior to deployment on the heart. More particularly, containment sheath 16 and delivery prongs 50 hold BACE device 100 in a closed or folded flower shape prior to deployment.

A small (approximately 2 inch) incision is made in the patient's thoracic region between the ribs. Barb fitting 24 of ball knob 22 is connected to a variable vacuum source so that suction cup 20 at the distal end of instrument 10 can provide variable levels of suction. The distal end of instrument 10 is then inserted through the incision into the patent and maneuvered until suction cup 20 is in contact with the desired location on the base of the heart. The vacuum source is then modulated until the desired amount of suction/grip on the base of the heart is achieved.

Once the base of the heart is engaged by suction cup 20, hollow shaft 12, delivery prongs 50, containment sleeve 16 and BACE device 100 are slid distally along center tube 14. Containment sleeve 16 is slid distally until inside the patient or as close as possible to the incision in the patient. In this embodiment, for the purposes of deploying BACE device 100 each wire slide 62 is coupled to its respective sleeve slide 64 in every slide pair 60. The surgeon grasps each slide pair 60 and slides them distally along longitudinal slots 13 of hollow shaft 12 until the distal ends of delivery prongs 50 and BACE device 100 emerge from containment sleeve 16.

The surgeon continues to slide delivery prongs 50 and BACE device 100 distally until BACE device 100 begins to expand from its compact folded flower shape into an expanded deployment shape. This expansion of BACE device 100 may be caused by outward bias of delivery prongs 50. For example, parallel guide wires 58 may be outwardly sprung so that as delivery prongs 50 emerge from the constraint of hollow shaft 12 and containment sleeve 16, delivery prongs 50 spread out and pull BACE device 100 into its expanded configuration. Alternatively, delivery prongs 50 may not be outwardly biased. Instead, they may be deflected into a spread open configuration, for example by sliding delivery prongs 50 along center tube 14 until the distal ends contact suction cup 20. Due to the conical shape of suction cup 22, delivery prongs 50 are deflected outward and cause BACE device 100 to spread open.

With BACE device 100 in an expanded deployment configuration on delivery prongs 50, the surgeon uses slide pairs 60 to maneuver BACE device 100 over the heart with fillable chambers 130 precisely aligned with predetermined areas on the heart that require treatment by increasing or decreasing pressure on those areas. To achieve such precise placement of BACE device 100, the surgeon can move each delivery prong 50 independently from every other delivery prong 50 by sliding only one of the slide pairs 60 at a time. Once BACE device 100 is placed in the desired location on the heart, one or more fillable chambers 130 is filled with a fluid via its respective fill tube 140. By selectively filling some fillable chambers 130 and not others, it is possible to provide precise pressure treatment to the outer surface of the heart. Areas immediately under a filled fillable chamber 130 will experience increased pressure or support. Areas between filled fillable chambers 130 will experience less pressure or support. By locating these areas of pressure and pressure relief in appropriate locations, the surgeon can improve heart valve function and prevent heart valve deterioration.

Once BACE device 100 is located on the heart as desired, BACE device 100 may be attached to the heart by suturing or a fastener prior to removal of delivery prongs 50 from BACE device 100 and prior to permanently filling fillable chambers 130. The surgeon may fill fillable chambers 130 to help locate and check placement and to make sure fillable chambers 130 are not folded, but tillable chambers 130 would then need to be deflated prior to attachment of BACE device 100 to the heart. Once the BACE device 100 is placed on the heart with fillable chambers 130 filled as desired, the surgeon begins the process of deploying BACE device 100 onto the heart and removing instrument 10 from the patient. One difficulty with removing instrument 10 from the patient is that BACE device 100 may tend to stick to delivery prongs 50 so that as instrument 10 is withdrawn from the patient, BACE device 100 is pulled slightly out of place. To solve this problem, each expandable wire member 52 is withdrawn inside its respective sleeve 54 prior to removing instrument 10 from the patient. To accomplish this, each wire slide 62 is decoupled from its respective sleeve slide 64 by lifting tab 65 of sleeve slide 64 over ridge 63 of wire slide 62. Once each slide pair 60 is decoupled in this manner, the surgeon slides one or more wire slides 62 proximally while keeping sleeve slides 64 stationary. By doing so, expandable wire member 52 begins to telescopically withdraw inside its respective sleeve 54. The curved distal end 55 of sleeve 54 is thereby brought into contact with the opening of pocket 150 as the friction between expandable wire member and the inner surface of pocket 150 causes pocket 150 to initially move in unison with expandable wire member 42. However, curved distal end 55 of sleeve 54 immediately halts movement of pocket 150 because curved distal end 55 of sleeve 54 is too large to fit into the entrance of pocket 150. As expandable wire member 52 telescopes inside sleeve 54, its width decreases due to the constraint of the inner walls of sleeve 54. Thus, expandable wire member 52 eventually withdraws completely outside of pocket 150 and into sleeve 54, while pocket 150 remains in the desired location on the heart, now completely detached from expandable wire member 52.

This process of withdrawing expandable wire member 52 from pocket 150 and into sleeve 54 is repeated for every delivery prong 50 until BACE device 100 is completely detached from instrument 10 and is deployed on the heart. At this point the vacuum source is turned off so that suction cup 20 no longer grips the base of the heart. The surgeon is then free to remove instrument 10 from the patient and complete the surgery.

The materials used to construct minimally invasive surgical instrument 10 are not critical, but the construction of an exemplary embodiment of instrument 10 will now be discussed. In one embodiment, hollow shaft 12 and center tube 14 are made from a metal such as anodized aluminum and stainless steel respectively. However, hollow shaft 12 and center tube 14 may also be made from other materials including polymers. Suction cup 20, sleeves 54, slide pairs 60 and ball knob 22 may all be made from a polymer such as polyvinylchloride (PVC), acrylonitrile butadiene styrene (ABS), polyethylene, or the like. Containment sheath 16 may be made from a soft flexible plastic such as polyethylene. Finally, expandable wire members 52 and parallel guide wires 58 may be made from any suitable metal or polymer including stainless steel, nitinol, or any other plastic or metal.

One of the main advantages of the disclosed embodiments is that the external basal annuloplasty device is passively frictionally held by the delivery prongs of the minimally invasive surgical instrument. This allows the external basal annuloplasty device to be deployed onto the heart without the requirement of disengaging a mechanism that is actively engaged with the external basal annuloplasty device. Another advantage of the disclosed embodiments is the ability to move each delivery prong independently of every other delivery prong. This enables the surgeon to place the external basal annuloplasty device on the heart with great precision. Similarly, another advantage provided by the disclosed embodiments is the ability to move each expandable wire member independently of its respective sleeve. This enables the surgeon to remove the surgical instrument from the patient without affecting the position of the external basal annuloplasty device because the sleeves prevent the external basal annuloplasty device from “sticking” to the expandable wire members when the surgeon begins sliding them proximally.

What has been described above includes examples of one or more embodiments. It is, of course, not possible to describe every conceivable combination of components or methodologies for purposes of describing the aforementioned embodiments, but one of ordinary skill in the art may recognize that many further combinations and permutations of various embodiments are possible. Accordingly, the described embodiments are intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A surgical instrument for delivering an external basal annuloplasty device to a heart, the surgical instrument comprising:
a hollow shaft having a proximal portion and a distal portion;
a center tube telescopically coupled to the hollow shaft and extending from the distal portion of the hollow shaft, the center tube having a heart gripping member at its distal end;
a plurality of delivery prongs slidably coupled to the hollow shaft, each delivery prong comprising:
an outwardly biased distal portion;
an expandable wire member within the outwardly biased distal portion for holding the external basal annuloplasty device;
a sleeve telescopically coupled to the expandable wire member;
a wire slide coupled to a proximal portion of the expandable wire member; and
a sleeve slide coupled to a proximal portion of the sleeve;
wherein the external basal annuloplasty device is mountable on the plurality of delivery prongs by inserting each expandable wire member into a respective pocket on the external basal annuloplasty device.

2. The surgical instrument of claim 1, wherein the wire slide and sleeve slide of each delivery prong are releasably coupled to each other, and wherein the external basal annuloplasty device is releasable from the plurality of delivery prongs by decoupling each sleeve slide from each respective wire slide and withdrawing each expandable wire member out of each respective pocket on the basal annuloplasty device and into each respective sleeve.

3. The surgical instrument of claim 1, wherein the expandable wire member of each delivery prong is an elongate flexible wire loop.

4. The surgical instrument of claim 3, wherein the elongate flexible wire loop is the distal end of a pair of substantially parallel guide wires with proximal ends coupled to the wire slide.

5. The surgical instrument of claim 3, wherein the elongate flexible wire loop increases in width when the elongate flexible wire loop is extended out of the sleeve and decreases in width when the elongate flexible wire loop is withdrawn into the sleeve.

6. The surgical instrument of claim 1, wherein the heart gripping member is a suction cup.

7. The surgical instrument of claim 6, wherein the center tube is hollow and connected to a vacuum source to provide a vacuum to the suction cup.

8. The surgical instrument of claim 1, wherein prior to delivery of the external basal annuloplasty device to the heart, the hollow shaft constrains the outwardly biased distal portions of the delivery prongs into a compact configuration substantially parallel to the hollow shaft

9. The surgical instrument of claim 1, wherein sliding each of the wire slides and sleeve slides distally along the hollow shaft causes the outwardly biased distal portions of the delivery prongs to outwardly expand into a deployment configuration.

10. The surgical instrument of claim 1, further comprising a containment sheath connected to the distal portion of the hollow shaft.

11. The surgical instrument of claim 1, further comprising an external basal annuloplasty device mounted on the plurality of delivery prongs, the external basal annuloplasty device comprising a plurality of pockets each receiving respective expandable wire members of the plurality of delivery prongs.

12. The surgical instrument of claim 1, wherein the external basal annuloplasty device comprises:
a band dimensioned to be received around a patient's heart, the band comprising an inner layer and an outer layer, wherein some but not all areas of the inner layer and outer layer are bound to one another; and
at least two tillable chambers in the band, the at least two fillable chambers being located in areas where the inner layer and the outer layer are not bound to one another;
wherein the at least two fillable chambers are positioned spaced apart from one another so as to form a bridge over vasculature on the heart when the at least two fillable chambers are filled.

13. A method of using the surgical instrument of claim 1 to deliver an external basal annuloplasty device to a heart in a minimally invasive surgical procedure, the method comprising:
providing the surgical instrument of claim 1;
providing an external basal annuloplasty device having a plurality of pockets;
mounting the external basal annuloplasty device on the surgical instrument by inserting each of the plurality of expandable wire members of the surgical instrument into respective pockets of the external basal annuloplasty device;
gripping the heart with the heart gripping member of the surgical instrument;
sliding the plurality of delivery prongs distally along the hollow shaft causing the outwardly biased distal portions of the delivery prongs and the external basal annuloplasty device to expand into a deployment configuration;
aligning the external basal annuloplasty device with a predetermined location on the heart; and
deploying the external basal annuloplasty device onto the heart by removing the plurality of expandable wire members of the surgical instrument from the respective pockets of the external basal annuloplasty device.

14. The method of claim 13, wherein removing the plurality of expandable wire members of the surgical instrument from the respective pockets of the external basal annuloplasty device is accomplished by decoupling each wire slide from each respective sleeve slide and sliding each wire slide proximally to withdraw each expandable wire member inside each respective sleeve.
